(19) United States
(12) Patent Application Publication
(10) Pub. No.: US 2022/0260521 A1
(43) Pub. Date: Aug. 18, 2022

(54) A PROCESS FOR SEPARATION AND QUANTITATION OF PROTEINS USING CAPILLARY ELECTROPHORESIS

(71) Applicant: KASHIV BIOSCIENCES, LLC, Bridgewater, NJ (US)

(72) Inventor: Roshan Ganeshlal UPADHYAY, Ahmedabad (IN)

(21) Appl. No.: 17/627,477

(22) PCT Filed: Jul. 14, 2020
(86) PCT No.: PCT/IB20/56593

§ 371 Date: Jan. 14, 2022

(30) Foreign Application Priority Data
Jul. 14, 2019 (IN) 201921028239

(51) Int. Cl. G01N 27/447

(52) U.S. Cl. CPC G01N 27/447; G01N 2333/535

(57) ABSTRACT

The present disclosure provides a method for analyzing, detecting and separating at least one low molecular weight impurity from a protein mixture using capillary electrophoresis, e.g., capillary electrophoresis-sodium dodecyl sulfate (CE-SDS). The present disclosure further provides methods to improve protein peak separation efficiency and quantification of a protein. Furthermore, the present disclosure provides an improved reduced CE-SDS method for analyzing a protein mixture comprising protein of interest which is pegylated and separates LMW or HMW fragments present in the protein mixture.

### FIELD OF DISCLOSURE

The present disclosure provides a method for analyzing, detecting and separating at least one low molecular weight impurity from a protein mixture using capillary electrophoresis, e.g., capillary electrophoresis-sodium dodecyl sulfate (CE-SDS). The present disclosure further provides methods to improve protein peak separation efficiency and quantification of a protein.

### BACKGROUND

Capillary electrophoresis-sodium dodecyl sulfate (CE-SDS) is known in the art to quantify protein fragments. In a typical CE-SDS process, proteins bound with SDS migrate through a gel-filled capillary in an electric field. The samples are denatured and reduced with suitable reducing agents, e.g., DTT, TCEP, 2-mercaptoethanol. The SDS binds with the reduced proteins in a constant ratio such that the magnitude of charge on each protein fragment containing SDS (protein—SDS fragment) is directly proportional to its molecular weight. The resulting protein—SDS fragments are introduced into the gel-filled capillary and migrate from the cathode (negative) towards the anode (positive). Small molecules migrate through the gel-filled capillary more rapidly than large molecules therefore, the protein—SDS fragments are detected in order of increasing molecular weight.

Reduced CE-SDS method is widely known and used for characterization of antibody heterogeneity produced through product-related impurities like low molecular weight (truncated antibody) or high molecular weight (dimer antibody). However, characterization and detection of low molecular weight (LMW) impurities in a protein mixture wherein the protein of interest is pegylated which does not comprise an antibody or fragment thereof, using reduced CE-SDS, is challenging and has not been used.

The present disclosure provides methods for the characterization and detection of low molecular weight (LMW) impurities and high molecular weight (HMW), in a non-antibody protein mixture, with reduced CE-SDS and Non-reduced CE-SDS. The present disclosure employs a reduced CE-SDS method to improve the quality and purity of biopharmaceutical products. The present disclosure provides a robust process to access and evaluate product quality and purity in order to make these properties compatible with the requirements of regulatory agencies.

Product-related impurities are considered critical quality attributes in therapeutic protein products and need to be monitored across the drug development process, including in QC release testing.

### SUMMARY OF THE INVENTION

The present disclosure provides an improved CE-SDS method of analyzing a protein mixture comprising protein of interest which is pegylated and does not contain an antibody (a non-antibody protein mixture) by separating LMW or HMW fragments present in the protein mixture.

The present disclosure provides an improved reduced CE-SDS method of analyzing a protein mixture comprising protein of interest which is pegylated and does not contain an antibody (a non-antibody protein mixture) by separating LMW or HMW fragments present in the protein mixture.

In certain embodiment the present invention provides a process for the quantification and/or detection of impurity in the protein mixture comprises;
a. Protein mixture comprising at least the impurity and the protein of interest, wherein the protein mixture does not contain an antibody;
b. Mixing the protein mixture with a suitable detergent;
c. Optionally incorporating a 10 kDa marker into the treated protein mixture;
d. Injecting the treated protein mixture in CE-instrument;
e. Performing CE SDS by injecting the treated protein mixture in a CE-instrument at suitable temperature; and
f. Detecting and/or quantifying the impurity;
wherein the impurity is selected from LMW impurity and HMW impurity.

In one aspect of this embodiment the present invention performs CE-SDS at capillary temperature below 25° C. In certain embodiment the capillary temperature is selected from 15° C., 16° C., 17° C., 18° C., 19° C., 20° C., 21° C. and 22° C. In certain embodiment the quantification and/or detection of low molecular weight or high molecular weight impurity is improved compared to CE-SDS performed at 25° C.

In one aspect of this embodiment the present invention provides protein mixture comprising protein of interest, optionally present impurity selected from LMW and HMW and suitable buffer or water.

In one aspect of this embodiment the present invention provides treated protein mixture comprises protein mixture treated with suitable buffer, detergent, optionally reducing agent capable of reducing noncovalent bonds in the protein mixture selected from DTT, B-mercaptoethanol and TCEP and optionally suitable marker having molecular weight selected from 10 kDa to 250 kDa. In certain embodiment marker's molecular weight helps in quantifying or detecting the LMW or HMW in protein mixture.

In certain embodiments, the disclosure provides a process for quantification and/or detection of an impurity having a molecular weight that is lower than the molecular weight of the protein of interest, in a treated protein mixture comprising:
a. Providing a protein mixture comprising at least the impurity and the protein of interest, wherein the protein mixture does not contain an antibody;
b. Mixing the protein mixture with a suitable detergent and reducing agent capable of reducing noncovalent bonds in the protein mixture;
c. Injecting the treated protein mixture in CE-instrument;
d. Performing CE SDS by injecting the treated protein mixture in a CE-instrument; and
e. Detecting and quantifying the impurity.

In certain embodiments, the disclosure provides a process for the quantification and/or detection of LMW impurity in a treated protein mixture comprising:
a. Providing a protein mixture comprising at least LMW impurity and the protein of interest;
b. Mixing the protein mixture with suitable buffer comprising o suitable detergent and reducing agent to form a treated protein mixture;
c. Injecting the treated protein mixture in a CE-instrument;
d. Performing the CE-SDS separation inside the instrument at below 25° C. to separate the LMW impurity from the protein of interest; and
e. Detecting the LMW impurity wherein quantification and/or detection of LMW impurity is improved compared to CE-SDS performed at 25° C.

In certain embodiments, the disclosure provides a process for the quantification and/or detection of LMW impurities in a treated protein mixture, comprising:
a. Providing a protein mixture comprising LMW impurity having a molecular weight of about 18 kDa, and the protein of interest having a molecular weight of about 38 kDa;
b. Mixing the protein mixture with suitable buffer, SDS and 2-mercaptoethanol to form a treated protein mixture;
c. Optionally incorporating a 10 kDa marker into the treated protein mixture
d. Injecting the treated protein mixture in a CE-instrument;
e. Performing the CE-SDS with capillary temperature below 25° C. to separate the LMW impurity from the protein of interest having a molecular weight of about 38 kDa; and
f. Detecting and quantifying the LMW impurity wherein quantification and/or detection of LMW impurity is improved compared to CE-SDS performed at 25° C.

In certain embodiments, the disclosure provides a process for the quantification and/or detection of PEG-GCSF comprising;
a. Preparing a protein mixture comprising the pegylated GCSF in suitable buffer or water;
b. Mixing the protein mixture with suitable buffer, SDS and reducing agent capable of reducing noncovalent bonds in the protein mixture to form a treated protein mixture;
c. Optionally incorporating a 10 kDa marker into the treated protein mixture;
d. Injecting the treated protein mixture in CE-instrument;
e. Performing the CE-SDS with keeping capillary temperature below 25° C.; and
f. Detecting the protein mixture to evaluate presence of unpegylated GCSF or fragment thereof;
wherein the CE-SDS provide improved quantification and/or detection of unpegylated GCSF or its fragment compared to CE-SDS performed at 25° C.

In certain embodiments, the disclosure provides a process for the analysis of PEG-GCSF comprising;
a. Preparing a protein mixture comprising the pegylated GCSF in suitable buffer or water;
b. Mixing the protein mixture with suitable buffer, SDS and reducing agent capable of reducing noncovalent bonds in the protein mixture to form a treated protein mixture;
c. Optionally incorporating a 10 kDa marker into the treated protein mixture;
d. Injecting the treated protein mixture in CE-instrument;
e. Performing the CE-SDS with keeping capillary temperature below 25° C.; and
f. Analyzing the protein mixture to evaluate presence of unpegylated GCSF or fragment thereof;
wherein the CE-SDS provides an improved analysis of unpegylated GCSF or its fragment compared to CE-SDS performed at 25° C.

In certain embodiments, the disclosure provides a method of performing CE-SDS at a separation voltage at about 15 kV or −15 kV for at least about 35 minutes.

In certain embodiments, the CE-SDS is performed at an injection voltage of about 5 kV or −5 kV for at least about 20 seconds.

### BRIEF DESCRIPTION OF THE FIGURES

FIG. 1 depicts overlay electropherograms of samples tested at different capillary temperatures. The migration time reduced with increasing the temperature of capillary. The peak shape is improved at 15° C. and 20° C. compared to 25° C.

FIG. 2 depicts Filgrastim (GCSF) peak elutes between 10 kDa and 20 kDa markers and PEG-Filgrastim (PEG-GCSF) main peak elutes between 35 kDa and 50 kDa molecular weight markers.

FIG. 3 depicts reduced CE-SDS electropherograms of diluent, formulation buffer, 10 kDa molecular weight marker, PEG-GCSF without 10 kDa marker, PEG-GCSF with 10 kDa marker, Neulasta (RMP). GCSF (filgrastim) and GCSF spiked with PEG-GCSF to determine if there are any matrix interference and proteinaceous interference peaks eluting in the region of integration of main peak, and LMW species. No interference peaks were observed in the region of integration of the peaks of interest, main peak and LMW species.

FIG. 4 depicts overlaid electropherograms of PEG_Filgrastim DS treated protein mixture with and without 10 kDa marker (Zoomed).

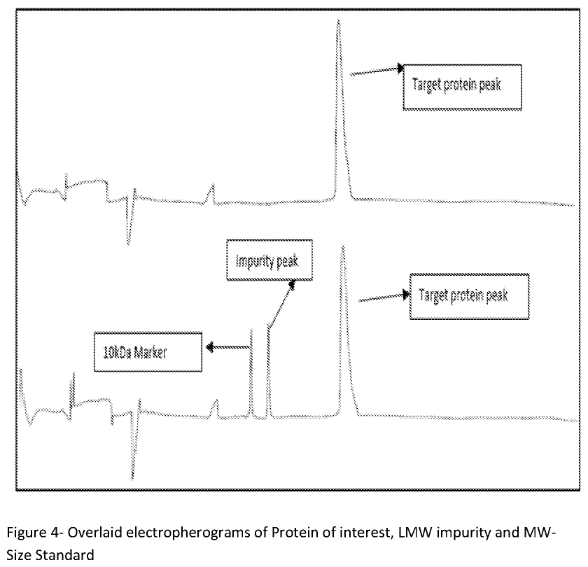

FIG. 5 depicts overlay electropherograms of samples tested at different sample preparation procedure and different incubation of treated protein mixture.

### DETAILED DESCRIPTION OF THE INVENTION

### Definitions

In order that the present disclosure can be more readily understood, certain terms are first defined. As used in this application, except as otherwise expressly provided herein, each of the following terms shall have the meaning set forth below. Additional definitions are set forth throughout the application.

As used herein, the term “protein of interest” is used in its broadest sense to include any protein (either natural or recombinant) with a desirable biological function. Such proteins of interest include, but are not limited to, enzymes, hormones, growth factors, cytokines and/or any fusion proteins PEGylated proteins, and any polymeric conjugate of the protein. In embodiment the protein of interest is pegylated GCSF having molecular weight about 38 kDa or 39 kDa.

The term about 38 kDa is interchangeable with 38 kDa or 39 kDa. The difference in the molecular weight is due to pegylation.

In certain embodiments, the protein of interest has a molecular weight of between about 10 kDa and about 150 kDa. In certain embodiments, the protein of interest has a molecular weight of between about e 25 kDa and about 150 kDa. In certain embodiments, the protein of interest has a molecular weight of at least about 38 kDa. In certain embodiments, the protein of interest is present in a mixture, for which purification is desired.

The terms “protein mixture” and “sample,” as used interchangeably herein, include a protein mixture comprises protein of interest and suitable buffer or water/aqueous solution and does not contain any antibody. In certain embodiment the protein mixture does not contain antibody and fragment thereof. In certain embodiments, the protein mixture comprises a plurality of proteins and protein of interest. In certain embodiments, the protein mixture optionally comprises low molecular weight impurity.

In certain embodiments, the protein mixture comprises of a protein having a molecular weight of between about 5 kDa and about 150 kDa. In certain embodiments, the impurity is a LMW impurity, a HMW impurity, an oxidized protein, or a deamidated protein.

The term “HMW species” refers to any one or more proteins with a higher apparent molecular weight relative to the intact protein of interest. The HMW species can be unrelated to the protein of interest or are aggregates, e.g., a dimer or multimer or any combination of the intact protein and any fragment thereof. In certain embodiments, the two proteins or protein fragments with different molecular weights may migrate together during electrophoresis because of non-uniform charge density due to non-uniform detergent coating of the proteins. In certain embodiment the HMW has molecular weight higher than about 38 kDa.

The term “LMW species” refers to one or more proteins with a lower apparent molecular weight relative to the intact protein of interest. The LMW species can be unrelated to the protein of interest or can be protein fragments. In certain embodiment the LMW has molecular weight lower than about 38 kDa. In one aspect of such embodiment the LMW is selected from 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa to 30 kDa. In certain embodiment the LMW is unpegylated GCSF. Unpegylated GCSF molecular weight is about 18 kDa. In certain embodiment the LMW is derived from pegylated GCSF or LMW is a fragment of Pegylated GCSF. In certain embodiment the LMW is present in protein mixture obtained from at least any one of previous steps of downstream, upstream, and stability. In certain embodiment the LMW is spiked in the protein mixture to verify the accuracy of detection of CE-SDS method. In certain embodiment the LMW spiked protein mixture and non-spiked LMW protein mixture are tested and compared to analyze the presence of LMW in non-spiked protein mixture.

The terms “treated protein mixture” include a protein mixture and detergent which is capable to denature the protein in mixture. In certain embodiment the detergent is sodium dodecyl sulphate (SDS). Optionally protein mixture is further treated with reducing agent capable of reducing the non-covalent bonds of proteins. Reducing agent is selected from DTT, B-mercaptoethanol and TCEP. Optionally protein mixture is further mixed with suitable marker having molecular weight selected from 10 kDa to 250 kDa. In certain embodiment marker's molecular weight helps in quantifying or detecting the LMW or HMW in protein mixture.

In certain embodiment the treated protein mixture comprises protein mixture mixed with detergent, reducing agent and optionally suitable marker.

The terms “separating” or “isolating,” as used interchangeably herein, refer to increasing the degree of purity of a protein of interest from a composition or sample comprising the protein of interest and one or more impurities. The separation or isolation can occur via electrophoresis, wherein an electric field is used to move negatively charged particles through a substance. When the particles have the same uniform electric charge, they migrate through the electric field based on their size, and the differential migration rates cause the particles, including proteins of interest, to be separated or isolated.

Capillary gel electrophoresis (CGE) using sodium dodecyl sulfate, commonly referred as CE-SDS, is the capillary format of polyacrylamide gel electrophoresis (SDS-PAGE), wherein the polyacrylamide slab gel is replaced with a narrow-bore glass capillary filled with a replaceable polymer sieving matrix. In contrast to traditional SDS-PAGE, CE-SDS can measure the overall fragmentation pattern and perform accurate protein quantitation with advantages including direct on-column UV or fluorescence detection, automation, and enhanced resolution and reproducibility.

SDS has been extensively utilized as the default detergent in both SDS-PAGE and CE-SDS techniques for protein denaturation, which can be attributed to a widely accepted consensus that protein tends to bind a relatively constant amount of SDS on a weight basis resulting in uniform mass/charge ratios of SDS-protein complexes in most cases. Consequently, intrinsic polypeptide charges of protein molecules become negligible and the final separation depends entirely on differences in the relative molecular mass of their denatured polypeptides.

The terms “detergent”, “surfactant” and “foaming agent” are used interchangeably herein. A detergent is a surfactant or a mixture of surfactants; when present in small amounts, a surfactant reduces the surface tension of a liquid (reduces the work needed to create the foam) or increases its colloidal stability by inhibiting coalescence of bubbles. A detergent reduces non-covalent interactions in proteins.

The term “quantification and/or detection” refers to proper peak separation obtained through CE-SDS for evaluation of fragments or size variants which are HMW or LMW compared to protein of interest. Proper peak separation does not have interference peak or peak broadening. Furthermore, peak separation is evaluated by parameters of Specificity, Linearity, Accuracy and Precision. In certain embodiment the quantification and/or detection is obtained through reduced CE-SDS.

The present disclosure provides a method of analyzing a protein mixture, which does not contain any antibody or any fragment thereof, by separating components of the protein mixture in reduced CE-SDS.

The present disclosure provides an improved CE-SDS method of analyzing a protein mixture comprising protein of interest which is pegylated and does not contain an antibody (a non-antibody protein mixture) by separating LMW or HMW fragments present in the protein mixture.

The present disclosure provides an improved reduced CE-SDS method of analyzing a protein mixture comprising protein of interest which is pegylated and does not contain an antibody (a non-antibody protein mixture) by separating LMW or HMW fragments present in the protein mixture.

In certain embodiment the present invention provides a process for the quantification and/or detection of impurity in the protein mixture comprises;
a. Protein mixture comprising at least the impurity and the protein of interest, wherein the protein mixture does not contain an antibody;
b. Mixing the protein mixture with a suitable detergent;
c. Optionally incorporating a 10 kDa marker into the treated protein mixture;
d. Injecting the treated protein mixture in CE-instrument;
e. Performing CE SDS by injecting the treated protein mixture in a CE-instrument at suitable temperature; and
f. Detecting and/or quantifying the impurity;
wherein the impurity is selected from LMW impurity and HMW impurity.

In one aspect of this embodiment the present invention performs CE-SDS at capillary temperature below 25° C. In certain embodiment the capillary temperature is selected from 15° C., 16° C., 17° C., 18° C., 19° C., 20° C., 21° C. and 22° C. In certain embodiment the quantification and/or detection of low molecular weight or high molecular weight impurity is improved compared to CE-SDS performed at 25° C.

In one aspect of this embodiment the present invention provides protein mixture comprising protein of interest, optionally present impurity selected from LMW and HMW and suitable buffer or water.

In one aspect of this embodiment the present invention provides treated protein mixture comprises protein mixture treated with suitable buffer, detergent, optionally reducing agent capable of reducing noncovalent bonds in the protein mixture selected from DTT, B-mercaptoethanol and TCEP and optionally suitable marker having molecular weight selected from 10 kDa to 250 kDa. In certain embodiment marker's molecular weight helps in quantifying or detecting the LMW or HMW in protein mixture.

In an embodiment the suitable buffer used in treated protein mixture is Tris buffer.

In an embodiment the preferred capillary temperature is below 21° C. In an embodiment the capillary temperature is about 15° C. to about 20° C. In preferred embodiment the capillary temperature is about 15° C. to about 17° C. In an embodiment the preferred capillary temperature is about 15° C.

In Pegylated proteins, PEG is attached to the protein and may interfere in elution with main peak. To reduce the impact of PEGylation, viscosity of separation conditions inside the capillary changed by changing the cartridge temperature. The effect of temperature on separation was evaluated and improved separation of peak is obtained at temperature at 20° C. and 15° C. compared to reduced CE-SDS performed at 25° C.

In an embodiment the present invention further treats protein mixture with reducing agent capable of reducing noncovalent bonds in the protein mixture selected from DTT, B-mercaptoethanol and TCEP.

In an embodiment the present invention provides protein mixture comprising protein of interest and suitable buffer or water and optionally impurity selected from LMW and HMW.

In an embodiment the present invention provides treated protein mixture comprises protein mixture treated with detergent, optionally reducing agent and optionally suitable marker having molecular weight selected from 10 kDa to 250 kDa. In certain embodiment marker's molecular weight helps in quantifying or detecting the LMW or HMW in protein mixture.

In such embodiment the detergent is SDS which is added into protein mixture with Tris buffer. In embodiment the SDS concentration is 1%.

In an embodiment the treated protein mixture comprises about 10 kDa marker which will be useful in quantifying or detect 18 kDa LMW impurity as about 18 kDa elutes after the marker peak of 10 kDa which assure the capability of CE-SDS process to resolve species having minor difference in their molecular weights.

In embodiment the protein of interest is pegylated GCSF having molecular weight about 38 kDa or 39 kDa.

In an embodiment the treated protein mixture is further centrifuged at about 10 k rpm to about 20 k rpm for about 10 to about 15 minutes at about 15° C. to about 25° C. and then incubated at about 55° C. to about 70° C. for 5 to 10 minutes. The treated protein mixture is cooled down at room temperature for 5 to 10 minutes and then finally centrifuged at 10 k rpm to about 20 k rpm for 10 to 20 minutes at 15° C. of temperature to about 70° C. before injecting into the CE-instrument.

In an embodiment the treated protein mixture is further centrifuged at about 10 k rpm for 10 minutes at 15° C. and then incubated at 55° C. for 5 minutes. The treated protein mixture is cooled down at room temperature for 5 minutes and then finally centrifuged at 10 k rpm for 10 minutes at 15° C. of temperature before injecting into the CE-instrument.

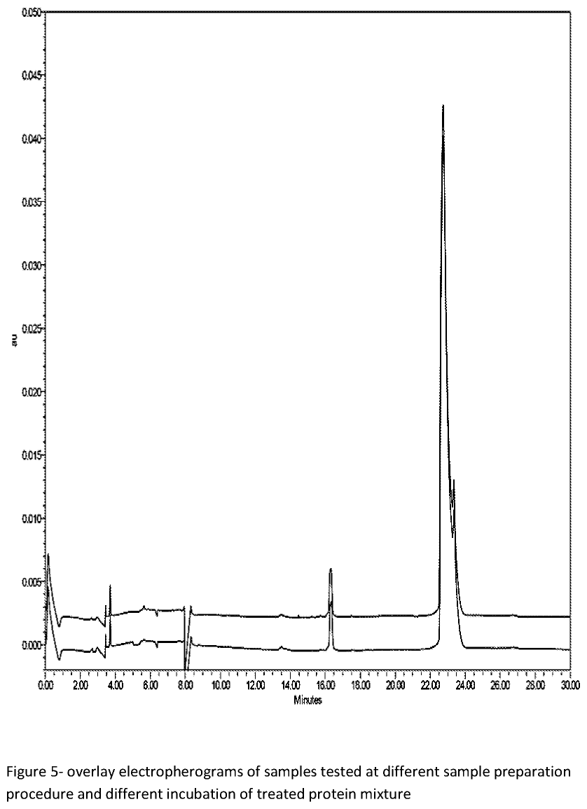

The present invention has shown the effect of sample preparation in FIG. 5 which is about 0.5 mg/mL and incubation of treated protein mixture at 60° C. and at 70° C.

In certain embodiments, the disclosure provides a process for quantification and/or detection of an impurity having a molecular weight that is lower than the molecular weight of the protein of interest in a treated protein mixture comprising:
a. Providing a protein mixture comprising at least the impurity and the protein of interest, wherein the protein mixture does not contain an antibody;
b. Mixing the protein mixture with a suitable buffer, detergent and reducing agent capable of reducing noncovalent bonds in the protein mixture;
c. Injecting the treated protein mixture in CE-instrument;
d. Performing CE SDS by injecting the treated protein mixture in a CE-instrument; and
e. Detecting and quantifying the impurity.

In certain embodiments, the disclosure provides a process for the quantification and/or detection of LMW impurity in a treated protein mixture, comprising:
a. Providing a protein mixture comprising at least LMW impurity and the protein of interest;
b. Mixing the protein mixture with a suitable buffer, detergent and reducing agent to form a treated protein mixture;
c. Injecting the treated protein mixture in a CE-instrument;
d. Performing the CE-SDS separation inside the instrument at below 25° C. to separate the LMW impurity from the protein of interest; and
e. Detecting the LMW impurity wherein quantification and/or detection of LMW impurity is improved compared to CE-SDS performed at 25° C.

In certain embodiment the LMW impurity is spiked into the protein mixture. In certain embodiment the LMW impurity is present in protein mixture from previous steps of upstream, downstream and stability.

In certain embodiments, the disclosure provides a process for the quantification and/or detection of LMW impurities in a treated protein mixture comprising:
a. Providing a protein mixture comprising LMW impurity having a molecular weight of about 18 kDa, and the protein of interest having a molecular weight of about 38 kDa;
b. Mixing the protein mixture with Tris buffer, SDS and 2-mercaptoethanol to form a treated protein mixture;
c. Optionally incorporating a 10 kDa marker into the treated protein mixture
d. Injecting the treated protein mixture in a CE-instrument;
e. Performing the CE-SDS with capillary temperature below 25° C. to separate the LMW impurity from the protein of interest having a molecular weight of about 38 kDa; and
f. Detecting and quantifying the LMW impurity wherein quantification and/or detection of LMW impurity is improved compared to CE-SDS performed at 25° C.

In certain embodiments, the disclosure provides a process for the quantification and/or detection of LMW impurities in a treated protein mixture comprising:
a. Providing a protein mixture comprising LMW impurity having a molecular weight of about 18 kDa, and the protein of interest having a molecular weight of about 38.8 kDa;
b. Mixing the protein mixture with Tris buffer, SDS and 2-mercaptoethanol to form a treated protein mixture;
c. Optionally incorporating a 10 kDa marker into the treated protein mixture
d. Injecting the treated protein mixture in a CE-instrument;
e. Performing the CE-SDS with capillary temperature below 25° C. to separate the LMW impurity from the protein of interest having a molecular weight of about 38.8 kDa; and
f. Detecting and quantifying the LMW impurity wherein quantification and/or detection of LMW impurity is improved compared to CE-SDS performed at 25° C.

In certain embodiments, the protein of interest has a molecular weight of between about 10 kDa to about 150 kDa. In certain embodiments, the protein of interest has a molecular weight of between from about 25 kDa to about 150 kDa.

In an embodiment the protein of interest is Pegylated protein and molecular weight is selected from about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 105 kDa, about 110 kDa, about 115 kDa, about 120 kDa, about 125 kDa, about 130 kDa, about 135 kDa, about 140 kDa, about 145 kDa, about 150 kDa.

The present invention is used for quantifying and/or detecting any impurity selected form LMW or HMW of protein of interest wherein the protein of interest is Pegylated protein and molecular weight is selected from about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 105 kDa, about 110 kDa, about 115 kDa, about 120 kDa, about 125 kDa, about 130 kDa, about 135 kDa, about 140 kDa, about 145 kDa, about 150 kDa.

In certain embodiments, the disclosure provides a process for the quantification and/or detection of PEG-GCSF comprising;
a. Preparing a protein mixture comprising the pegylated GCSF in suitable buffer or water;
b. Mixing the protein mixture with suitable buffer, SDS and reducing agent capable of reducing noncovalent bonds in the protein mixture to form a treated protein mixture;
c. Optionally incorporating a 10 kDa marker into the treated protein mixture;
d. Injecting the treated protein mixture in CE-instrument;
e. Performing the CE-SDS with keeping capillary temperature below 25° C.;
f. Detecting the protein mixture to evaluate presence of unpegylated GCSF;
wherein the CE-SDS provide improved quantification and/or detection of unpegylated GCSF compared to CE-SDS performed at 25° C.

In certain embodiments, the disclosure provides a process for the analysis of PEG-GCSF comprising;
a. Preparing a protein mixture comprising the pegylated GCSF in suitable buffer or water;
b. Mixing the protein mixture with Tris buffer, SDS and B-mercaptoethanol to form a treated protein mixture;
c. Optionally incorporating a 10 kDa marker into the treated protein mixture;
d. Injecting the treated protein mixture in CE-instrument;
e. Performing the CE-SDS with keeping capillary temperature below 25° C.; and
f. Analyzing the protein mixture to evaluate presence of unpegylated GCSF or fragment thereof;
wherein the CE-SDS provide improved analysis of unpegylated GCSF compared to CE-SDS performed at 25° C.

In certain embodiments, the disclosure provides a method of performing CE-SDS at a separation voltage at about 15 kV for at least about 35 minutes.

In certain embodiments, the CE-SDS is performed at an injection voltage of about 5 kV for at least about 20 seconds. In certain embodiments, the CE-SDS is performed with Tris buffer or HEPES buffer.

In certain embodiments, the CE-SDS is performed at a pH of between about 6 and about 10. In certain embodiments, the CE-SDS is performed with Tris buffer at pH 9.

In certain embodiments, the CE-SDS is performed at separation voltage at about 15 kV, for at least about 30 minutes. In certain embodiments, the CE-SDS is performed at a separation voltage of about 15 kV, for at least about 35 minutes.

The migration time of eluting peaks depends on separation voltage which also helps in resolving the peaks. The presence invention is not bound to use of separation voltage 15 kV as the optimizing the separation voltage and its respective effect on the elution of the peaks are well known to the skilled person.

In certain embodiments, the CE-SDS is performed at an injection voltage of about 5 kV for at least about 10 seconds. In certain embodiments, the CE-SDS is performed at an injection voltage of about −5 kV, between about 0 and about 15 seconds.

In certain embodiments, the treated protein mixture is injected at a room temperature of about 25° C.

In certain embodiments, the capillary temperature is below 25° C. In certain embodiments, the capillary temperature is between about 15° C. and about 24° C. In certain embodiments, the capillary temperature is between about 15° C. and about 20° C. In certain embodiments, the capillary temperature is between about 15° C. and about 18° C. In certain embodiments, the capillary temperature is about 15° C.

In certain embodiments, the protein mixture does not contain any antibody or a fragment thereof. In certain embodiments, the protein mixture does not contain a full-length antibody having a molecular weight of about 150 kDa but can contain an antibody fragment having a molecular weight of less than about 100 kDa.

In certain embodiments, the protein mixture comprises a fusion protein comprising an antibody fragment. In certain embodiments, the protein mixture comprises a fusion protein comprising an antibody fragment attached with PEG.

In certain embodiments, the disclosure provides a process for the analysis of PEG-GCSF comprising;
a. Preparing a protein mixture comprising the pegylated GCSF in suitable buffer or water;
b. Mixing the protein mixture with Tris buffer, SDS to form a treated protein mixture;
c. Optionally incorporating a 10 kDa marker into the treated protein mixture;
d. Injecting the treated protein mixture in CE-instrument;
e. Performing the CE-SDS with keeping capillary temperature at about 25° C.; and
f. Analyzing the protein mixture to evaluate presence of unpegylated GCSF.

### Preparation of Protein Mixture and Treated Protein Mixture

The protein mixture comprises protein of interest and suitable buffer or water/aqueous solution and does not contain any antibody. In certain embodiment the protein mixture does not contain antibody and fragment thereof. In certain embodiments, the protein mixture comprises a plurality of proteins and protein of interest. The plurality of proteins is selected from fragments and protein except protein of interest. It may form LMW or HMW impurity. In certain embodiments, the protein mixture optionally comprises low molecular weight impurity.

In certain embodiment the LMW is present in protein mixture obtained from at least any one of previous steps of downstream, upstream, and stability. In certain embodiment the LMW is spiked in the protein mixture to verify the accuracy of detection of CE-SDS method. In certain embodiment the LMW spiked protein mixture and non-spiked LMW protein mixture are tested and compared to analyze the presence of LMW in non-spiked protein mixture.

In an embodiment the protein mixture is at least about 2 mg/ml. In certain embodiment the protein mixture is selected from about 2 mg/ml, about 3 mg/ml, about 4 mg/ml, about 5 mg/ml. In an embodiment the protein mixture is at least about 5 mg/ml.

In certain embodiments, the treated protein mixture is prepared by mixing a protein mixture in a suitable buffer comprising a detergent, and a suitable reducing agent. In certain embodiments, the reducing agent is DTT, B-mercaptoethanol, or TCEP. In certain embodiments, the selection of reducing agent depends on the protein of interest present in the protein mixture.

The suitable buffer is Tris buffer at pH 9. Tris concentration is selected from 60 mM to 90 mM.

In certain embodiments, the treated protein mixture comprises an aqueous or buffer solution containing a protein mixture optionally having at least one LMW impurity, a protein of interest, SDS, and B-mercaptoethanol. In certain embodiments, the treated protein mixture further comprises a marker having a molecular weight that is lower than the targeted impurity or LMW impurity, for minimizing the uncertainty of peak shift. In certain embodiments, the treated protein mixture comprises a marker having a molecular weight of about 10 kDa.

In certain embodiments, the present disclosure provides a process for separation of a pegylated granulocyte colony stimulating factor (GCSF), the process comprising:
a. Providing a protein mixture comprising a GCSF or fragments thereof, and PEG-GCSF having a molecular weight of about 38 kDa;
b. Mixing the protein mixture with Tris, SDS and 2-mercaptoethanol to form a treated protein mixture;
c. Optionally, incorporating a 10 kDa marker into the treated protein mixture;
d. Injecting the treated protein mixture in CE-SDS;
e. Performing the CE-SDS with capillary temperature below 25° C. to separate the GCSF or fragment thereof from the PEG-GCSF; and
f. Separating the GCSF or fragments thereof from the PEG-GCSF wherein the separation is improved compared to CE-SDS performed at 25° C.

In certain embodiments, the present disclosure provides a process for improving protein peak separation efficiency in CE-SDS, comprising:
a. Providing a protein mixture comprising a LMW impurity having a molecular weight of at least about 18 kDa, and a protein of interest having a molecular weight of about 38 kDa;
b. Mixing the protein mixture with Tris, SDS and 2-mercaptoethanol to form a treated protein mixture;
c. Injecting the treated protein mixture in CE-SDS;
d. Performing the CE-SDS with capillary temperature below 25° C. to separate the LMW impurity from the protein having a molecular weight of about 38 kDa; and
e. Separating the LMW impurity from the protein having a molecular weight of about 38 kDa through CE-SDS wherein the separation is improved compared to CE-SDS performed at 25° C.

In certain embodiments, the present disclosure provides a process for improving protein peak separation efficiency in CE-SDS, comprising:
a. Providing a protein mixture comprising LMW impurity having a molecular weight of at least about 18 kDa, and a protein of interest having a molecular weight of about 38 kDa;
b. Mixing the protein mixture with SDS, 2-mercaptoethanol, and a 10 kDa marker to form a treated protein mixture;
c. Performing a centrifugation at about 10K rpm at 15° C. and then incubated at 55° C. for about 5 minutes.
d. Cooling down the treated protein mixture at room temperature for 5 minutes and optionally further centrifuged at 10 k rpm for 10 minutes at 15° C.
e. Injecting the treated protein mixture in CE-SDS;
f. Performing the CE-SDS to separate the LMW impurity from the protein of interest having a molecular weight of about 38 kDa; wherein the capillary temperature is about 15(+5°) C. and the separation voltage used is at least about 15 kV
g. Separating the LMW impurity from the protein of interest having a molecular weight of about 38 kDa using CE-SDS.

In certain embodiments, the present disclosure provides a process for improving protein peak separation efficiency in CE-SDS comprising:
a. Providing a protein mixture comprising at least LMW impurity and a protein of interest having a molecular weight of at least 25 kDa, wherein the protein mixture does not have an antibody and a fragment thereof;
b. Mixing the protein mixture with SDS and 2-mercaptoethanol to form a treated protein mixture;
c. Optionally, adding a 10 kDa marker to the treated protein mixture;
d. Injecting the treated protein mixture into CE-SDS;
e. Performing the CE-SDS to separate the LMW impurity from the protein of interest having a molecular weight of about 38 kDa; and
f. Separating the LMW impurity from the desired protein of interest having a molecular weight of at least about 25 kDa through CE-SDS.
Wherein the CE-SDS capillary temperature is below 25° C. and thereby the CE-SDS improves protein peak separation efficiency compared to CE-SDS performed at 25° C.

In certain embodiment, the disclosure provides a process for improving protein peak separation efficiency in CE-SDS and thereby is capable to separate a LMW impurity from the protein of interest, the process comprising:
a. Providing a protein mixture comprising an 18 kDa LMW impurity, and a protein of interest having a molecular weight of about 38 kDa;
b. Mixing the protein mixture with Tris, SDS, 2-mercaptoethanol and, optionally, a 10 kDa marker to form the treated protein mixture;
c. Injecting the treated protein mixture in CE-SDS;
d. Performing the CE-SDS to separate the LMW impurity from the protein of interest having a molecular weight of about 38 kDa;
e. Separating the LMW impurity from the protein having a molecular weight of about 38 kDa using CE-SDS; and
Wherein the CE-SDS separation condition comprises setting the injection voltage to at least about −5 kV for about 15 seconds, setting the sample temperature to about 10° C., and setting the capillary temperature to about 15 (+5) ° C. wherein the separation is improved compared to CE-SDS performed at 25° C.

In certain embodiments, the disclosure provides a process for the quantification and/or detection of PEG-GCSF comprising;
a. Preparing a protein mixture comprising the pegylated GCSF in suitable buffer or water;
b. Mixing the protein mixture with Tris, SDS and reducing agent capable of reducing noncovalent bonds in the protein mixture to form a treated protein mixture;
c. Optionally incorporating a 10 kDa marker into the treated protein mixture;
d. Injecting the treated protein mixture in CE-instrument;
e. Performing the CE-SDS with keeping capillary temperature below 25° C.; and
f. Detecting the protein mixture to evaluate presence of unpegylated GCSF or fragment thereof;
wherein the CE-SDS provide improved quantification and/or detection of unpegylated GCSF compared to CE-SDS performed at 25° C.

In certain embodiments, the disclosure provides a process for the quantification and/or detection of PEG-GCSF comprising;
a. Preparing a protein mixture comprising the pegylated GCSF in suitable buffer or water;
b. Mixing the protein mixture with Tris, SDS and reducing agent capable of reducing noncovalent bonds in the protein mixture to form a treated protein mixture;
c. Optionally incorporating a 10 kDa marker into the treated protein mixture;
d. Injecting the treated protein mixture in CE-instrument;
e. Performing the CE-SDS with keeping capillary temperature is 15° C.; and
f. Detecting the protein mixture to evaluate presence of unpegylated GCSF or fragment thereof;
wherein the CE-SDS provide improved quantification and/or detection of unpegylated GCSF compared to CE-SDS performed at 25° C.

In certain embodiments, the disclosure provides a process for the analysis of PEG-GCSF comprising;
a. Preparing a protein mixture comprising the pegylated GCSF in suitable buffer or water;
b. Mixing the protein mixture with Tris buffer, SDS and reducing agent capable of reducing noncovalent bonds in the protein mixture to form a treated protein mixture;
c. Optionally incorporating a 10 kDa marker into the treated protein mixture;
d. Injecting the treated protein mixture in CE-instrument;
e. Performing the CE-SDS with keeping capillary temperature below 25° C.; and
f. Analyzing the protein mixture to evaluate presence of unpegylated GCSF or fragment thereof;
wherein the CE-SDS provide improved analysis of unpegylated GCSF compared to CE-SDS performed at 25° C.

In certain embodiments, the disclosure provides a process for the analysis of PEG-GCSF comprising;
a. Preparing a protein mixture comprising the pegylated GCSF in suitable buffer or water;
b. Mixing the protein mixture with Tris buffer, SDS and reducing agent capable of reducing noncovalent bonds in the protein mixture to form a treated protein mixture;
c. Optionally incorporating a 10 kDa marker into the treated protein mixture;
d. Injecting the treated protein mixture in CE-instrument;
e. Performing the CE-SDS with keeping capillary temperature is 15° C.; and
f. Analyzing the protein mixture to evaluate presence of unpegylated GCSF or fragment thereof;
wherein the CE-SDS provide improved analysis of unpegylated GCSF compared to CE-SDS performed at 25° C.

The present invention provides examples for illustration purpose only, and the scope of the invention should not be considered limited by the presented examples.

### Example 1

Pegfilgrastim is generated by covalently attaching a 20 kDa polyethylene glycol (PEG) moiety to the amino terminus of filgrastim produced in Escherichia coli. Pegfilgrastim keeps the same biological activity as filgrastim but has a longer circulating half-life. Pegfilgrastim has a total molecular weight of 39,000 Daltons and is indicated for reducing the incidence of infection.

For the reduced CE-SDS method for Peg-filgrastim, the Specificity was assessed from injection of diluent (water), formulation buffer, and oxidized Peg-filgrastim sample. Electropherograms of diluent and formulation buffer are evaluated to determine if there are any proteinaceous interference peaks eluting in the region of integration of main peak, and LMW species. No interference peaks are observed in the region of integration of the peaks of interest, main peak, and the LMW species.

### Evaluation of Cartridge Temperature and Sample Preparation

The initial separation conditions for CE-SDS consist of separation voltage (−15 kV for 35 or 40 minutes), injection voltage (−5 kV for 20 seconds), PDA detector wavelength (220 nm), sample temperature (25° C.), and capillary temperature (25° C.). Since the PEG attached in Pegylated protein might be interfering in the elution of main peak. To reduce the impact of PEGylation, viscosity of separation conditions inside the capillary changed by changing the cartridge temperature. The effect of temperature on separation is evaluated by changing the capillary temperature from 25° C. to 20° C. and 15° C.

Use of master mix was also removed by adding each component (sample buffer, 10 kDa marker and 2-mercaptoethanol) individually in the sample preparation. As it is known that Peg-filgrastim (˜12.5 mg/mL) and its syringe (˜10 mg/mL) have a much higher concentration than 0.50 mg/mL, the sample is prepared as 5 mg/mL in DI water initially and then 10 μL of 5 mg/mL sample added into 83 μL of sample buffer, mixed it well. After that 2 μL of 10 kDa marker and 5 μL of 2-mercaptoethanol are added into the solution. The sample is centrifuged at 14 k rpm for 10 minutes at 15° C. of temperature and then incubated at 55° C. for 5 minutes. The sample is then cooled down at room temperature for 5 minutes and then finally centrifuged at 14 k rpm for 10 minutes at 15° C. of temperature before the analysis.

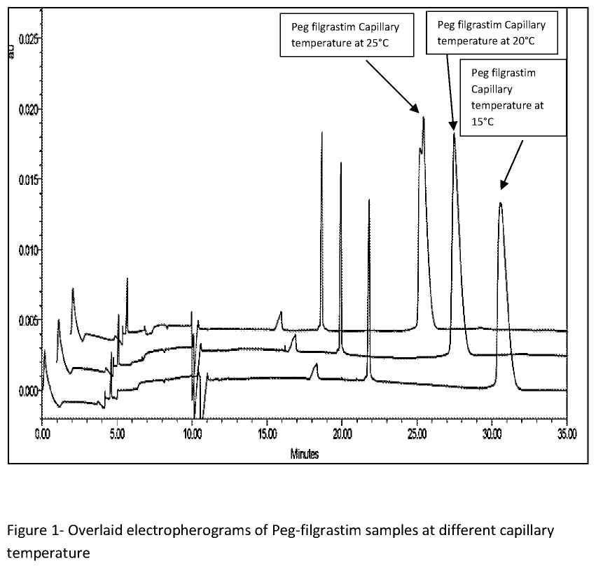

FIG. 1 shows the overlay electropherograms of samples tested at different capillary temperatures. The migration time reduced with increasing the temperature of capillary. The peak shape is improved as the temperature is decreased from 25° C. to 15° C.

Based on the FIG. 1, 15° C. temperature is more suitable for the method.

### Evaluation of Molecular Weight

To check the migration time for Peg-filgrastim and filgrastim peaks with respect to their molecular weights, protein molecular weight size standard was injected along with filgrastim and Peg-filgrastim processed samples.

The protein size standard contains seven proteins of 10, 20, 35, 50, 100, 150, and 225 kDa. The Peg-filgrastim (˜39 kDa) peak should elute between peak 3 and peak 4 having molecular weights of 35 and 50 respectively.

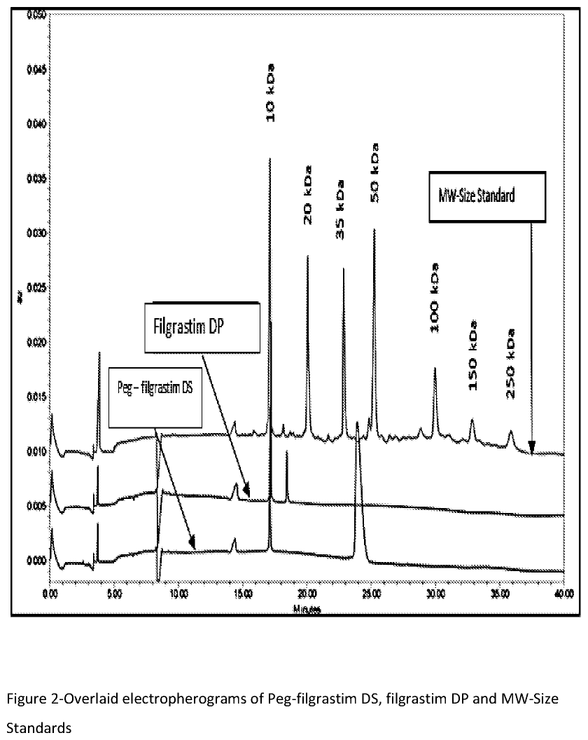

As FIG. 2 shows, the filgrastim peak is eluting between 10 kDa and 20 kDa markers and Peg-filgrastim main peak is eluting between 35 kDa and 50 kDa molecular weight markers. It confirms that the filgrastim peak and Peg-filgrastim main peak are eluting with respect to their molecular size and shape.

The use of a 10 kDa molecular weight marker is employed as an internal control, mainly to check the starting window of integration since filgrastim elutes after the marker peak and also to check the method capability to resolve species having minor difference in their molecular weights. In this case marker peak having 10 kDa molecular weight and filgrastim peak which is having ˜18 kDa molecular weight.

Since the concentration of Peg-filgrastim DP is ˜10 mg/mL and DS is more than 10 mg/mL, the sample preparation step starts with making of 5 mg/mL of stock sample using DI water or equivalent. After that sample is prepared by adding 10 μL of 5 mg/mL sample into 83 μL of sample buffer, 2 μL of 10 kDa marker, and 5 μL of 2-mercaptoethanol to make the final volume of 100 μL (The component of sample preparation may change proportionally, according to the final volume).

The sample is centrifuged at 14 k rpm for 10 minutes at 15° C. of temperature and then incubate at 55° C. for 5 minutes. The sample is cooled down at room temperature for 5 minutes and then finally centrifuge at 14 k rpm for 10 minutes at 15° C. of temperature before the analysis.

Based on the selected conditions, the method performance was evaluated. The following parameters were investigated, Specificity, linearity, accuracy, precision, and the LOQ & LOD determined from a spiked sample.

TABLE 1
Time Program in Separation Method

| Time | | | | | Inlet | Inlet | Outlet | Outlet |
|---|---|---|---|---|---|---|---|---|
| (min) | Event | Value | Duration | | vial | tray | vial | tray |
| | Rinse | 4854.1 | 3.00 | min | D1 | Buffer | D1 | Buffer |
| | pressure | | | | | | | |
| | Rinse | 4854.1 | 1.00 | min | E1 | Buffer | E1 | Buffer |
| | pressure | | | | | | | |
| | Rinse | 4854.1 | 1.00 | min | F1 | Buffer | F1 | Buffer |
| | pressure | | | | | | | |
| | Rinse | 4854.1 | 10.00 | min | B1 | Buffer | B1 | Buffer |
| | pressure | | | | | | | |
| | Wait | | 0.00 | min | A1 | Buffer | A1 | Buffer |
| | Wait | | 0.00 | min | A4 | Buffer | A4 | Buffer |
| | Inject | −5.0 kV | 15.0 | S | A0 | Buffer | C1 | Buffer |
| | Voltage | | | | | | | |
| | Wait | | 0.00 | min | B4 | Buffer | B4 | Buffer |
| 0.00 | Separation | −15.0 kV | 35.00 | min | C1 | Buffer | C1 | Buffer |
| | Voltage | | | | | | | |
| 5.00 | Autozero | | | | | | | |
| 35.00 | End | | | | | | | |

### Example 2

Specificity refers to the ability to assess unequivocally the analyte in the presence of components which may be expected to be present. Specificity of method should be assessed by checking matrix interference and by evaluating separation selectivity of the method.

For reduced CE-SDS method for PEG-Filgrastim, the Specificity was assessed from:
1. DI water (Blank)—To check the matrix interference
2. Formulation buffer of Peg-filgrastim—To check the matrix interference
3. 10 kDa molecular weight marker—To check the matrix interference
4. Peg-filgrastim DS without 10 kDa marker—To check the separation selectivity
5. Peg-filgrastim DS with 10 kDa marker (control)—To check the separation selectivity
6. Neulasta (RMP)—To check the separation selectivity
7. filgrastim—To check the separation selectivity
8. DS spiked with filgrastim—To check the separation selectivity

Electropherograms of diluent and formulation buffer were evaluated to determine if there are any proteinaceous interference peaks eluting in the region of integration of main peak, and LMW species. No interference peaks were observed in the region of integration of the peaks of interest, main peak and LMW species.

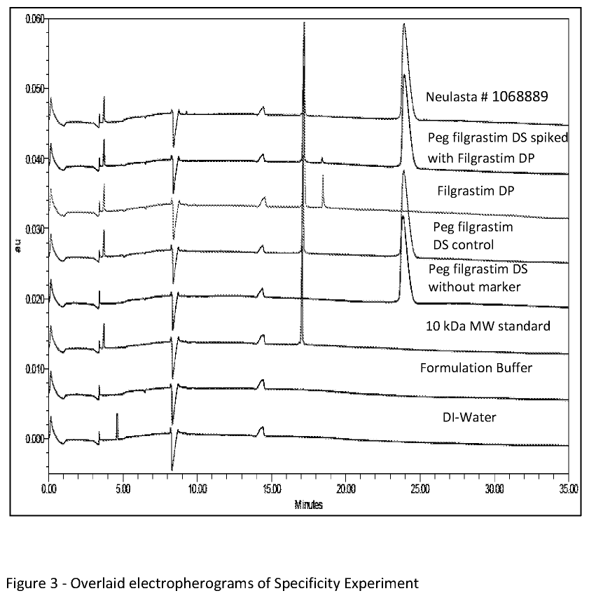

FIG. 3 show Electropherograms of all the aforementioned components. The 10 kDa marker peak is very well separated from the peak of GCSF and the GCSF peak is well separated from the Peg-filgrastim peak. This proves that the method has separation selectivity and the resolution of the method is also at par to resolve the peaks.

### Example 3

Non-Reduced CE-SDS Method Description for PEG-GCSF

The sample preparation step starts with making of 1.5 mg/mL of stock sample using DI water or equivalent. After that sample is prepared by adding 15 μL of 1.5 mg/mL sample into 95 μL of master mix, which contains 64 μL of sample buffer, 29.5 μL of DI water and 1.5 μL of 10 kDa marker. The sample is centrifuged at 14 k rpm for 10 minutes at 15° C. of temperature and then incubate at 55° C. for 5 minutes. The sample is cooled down at room temperature for 5 minutes and then finally centrifuge at 14.8 k rpm for 5 minutes at ambient temperature before the analysis.

Capillary temperature: 25° C.

TABLE 2
Time Program in Separation Method

| Time | | | | | Inlet | Inlet | Outlet | Outlet |
|---|---|---|---|---|---|---|---|---|
| (min) | Event | Value | Duration | | vial | tray | vial | tray |
| | Rinse | 4854.1 | 3.00 | min | D1 | Buffer | D1 | Buffer |
| | pressure | | | | | | | |
| | Rinse | 4854.1 | 1.00 | min | E1 | Buffer | E1 | Buffer |
| | pressure | | | | | | | |
| | Rinse | 4854.1 | 1.00 | min | F1 | Buffer | F1 | Buffer |
| | pressure | | | | | | | |
| | Rinse | 4854.1 | 10.00 | min | B1 | Buffer | B1 | Buffer |
| | pressure | | | | | | | |
| | Wait | | 0.00 | min | A1 | Buffer | A1 | Buffer |
| | Wait | | 0.00 | min | A4 | Buffer | A4 | Buffer |
| | Inject | −5.0 kV | 20.0 | S | A0 | Buffer | C1 | Buffer |
| | Voltage | | | | | | | |
| | Wait | | 0.00 | min | B4 | Buffer | B4 | Buffer |
| 0.00 | Separation | −15.0 kV | 30.00 | min | C1 | Buffer | C1 | Buffer |
| | Voltage | | | | | | | |
| 5.00 | Autozero | | | | | | | |
| 30.00 | End | | | | | | | |

We claim:

1. A process for the quantification and/or detection of impurity in the protein mixture comprises;
a. Protein mixture comprising at least the impurity and the protein of interest, wherein the protein mixture does not contain an antibody;
b. Mixing the protein mixture with a suitable buffer and detergent;
c. Optionally incorporating a 10 kDa marker into the treated protein mixture;
d. Injecting the treated protein mixture in CE-instrument;
e. Performing CE SDS by injecting the treated protein mixture in a CE-instrument at suitable temperature; and
f. Detecting and/or quantifying the impurity;
wherein the impurity is selected from LMW impurity and HMW impurity.

2. The process according to claim 1 further comprises mixing the protein mixture in step (b) with reducing agent capable of denaturing the protein and reducing noncovalent bonds in the protein mixture.

3. The process according to claim 2 wherein the reducing agent is selected from DTT, B-mercaptoethanol and TCEP.

4. The process according to claim 1 wherein the detergent is sodium dodecyl sulphate or SDS.

5. The process according to claim 1 wherein the SDS concentration is 1%.

6. The process according to claim 1 wherein the protein mixture further comprises water or buffer.

7. The process according to claim 1 wherein the protein mixture is at least 2 mg/ml.

8. The process according to claim 7 wherein the protein mixture is at least 2 mg/ml to 5 mg/ml.

9. The process according to claim 1 wherein the protein mixture does not comprise an antibody and/or fragment thereof.

10. The process according to claim 1 wherein the suitable temperature of CE-SDS capillary is below 25° C.

11. The process according to claim 10 wherein the capillary temperature is selected from 15° C., 16° C., 17° C., 18° C., 19° C., 20° C., 21° C., 22° C.

12. The process according to claim 11 wherein the capillary temperature is 15° C.

13. The process according to claim 1 wherein the quantification or detection of low molecular weight or high molecular weight impurity is improved compared to CE-SDS performed at 25° C.

14. The process according to claim 1 wherein the CE-SDS is performed at separation voltage at about 15 kV or −15 kV for at least about 30 minutes.

15. The process according to claim 1 wherein the CE-SDS is performed at an injection voltage of about 5 kV or −5 kV for at least about 10 seconds.

16. The process according to claim 1 wherein the CE-SDS is performed with Tris buffer at pH 9.

17. The process according to claim 1 wherein the suitable buffer is Tris.

18. The process according to claim 16 wherein the Tris concentration is selected from 60 mM to 90 mM.

19. The process according to claim 1 wherein the low molecular weight impurity is less than about 38 KDa.

20. The process according to claim 1 wherein the low molecular weight impurity is GCSF or any other fragments derived from PEG-GCSF.

21. The process according to claim 1 wherein the high molecular weight impurity is higher than about 38 KDa.

22. A process for an improving protein peak separation efficiency in CE-SDS comprising:
a. Providing a protein mixture comprising a LMW impurity having a molecular weight of at least about 18 kDa, and a protein of interest having a molecular weight of about 38 kDa;
b. Mixing the protein mixture with Tris, SDS and 2-mercaptoethanol to form a treated protein mixture;
c. Injecting the treated protein mixture in CE-SDS;
d. Performing the CE-SDS with capillary temperature below 25° C. to separate the LMW impurity from the protein having a molecular weight of about 38 kDa; and
e. Separating the LMW impurity from the protein having a molecular weight of about 38 kDa through CE-SDS
wherein the separation is improved compared to CE-SDS performed at 25° C.

23. A process for the quantification and/or detection of GCSF in the protein mixture comprises;
a. Protein mixture comprising the GCSF, pegylated GCSF and suitable buffer or water;
b. Mixing the protein mixture with Tris buffer, SDS and 2-mercaptoethanol to form a treated protein mixture;
c. Optionally incorporating a 10 kDa marker into the treated protein mixture;
d. Injecting the treated protein mixture in CE-instrument;
e. Performing the CE-SDS to separate the GCSF from pegylated GCSF; and
f. Detecting and/or quantifying the GCSF;
wherein the CE-SDS provide improved quantification and/or detection of GCSF or any other fragment compared to CE-SDS performed at 25° C.

24. A process for the analysis of PEG-GCSF comprising;
a. Preparing a protein mixture comprising the pegylated GCSF in suitable buffer or water;
b. Mixing the protein mixture with Tris buffer, SDS and 2-mercaptoethanol to form a treated protein mixture;
c. Optionally incorporating a 10 kDa marker into the treated protein mixture;
d. Injecting the treated protein mixture in CE-instrument;
e. Performing the CE-SDS with keeping capillary temperature below 25° C.; and
f. Analyzing the protein mixture to evaluate presence of unpegylated GCSF;
wherein the CE-SDS provide improved analysis of unpegylated GCSF compared to CE-SDS performed at 25° C.
